(19) United States
(12) Patent Application Publication
(10) Pub. No.: US 2026/0202401 A1
(43) Pub. Date: Jul. 16, 2026

(54) DUAL PAD COMPOSITIONS AND ASSOCIATED DEVICES AND METHODS FOR DIRECT COLLECTION OF A FIXED SPECIMEN VOLUME FOR USE WITH LATERAL FLOW ASSAYS

(71) Applicant: InBios International, Inc., Seattle, WA (US)

(72) Inventor: James William Needham, Seattle, WA (US)

(21) Appl. No.: 19/446,731

(22) Filed: Jan. 12, 2026

Related U.S. Application Data
(60) Provisional application No. 63/744,624, filed on Jan. 13, 2025.

(51) Int. Cl. G01N 33/543

(52) U.S. Cl. CPC G01N 33/54388

(57) ABSTRACT

The compositions and methods described herein relate to the direct collection of a fixed specimen volume using a dual pad comprising a first hydrophilic sample pad and a second hydrophobic sample pad. The fixed specimen volume collected on the first hydrophilic sample pad is prevented, or blocked, from saturating anything other than the hydrophilic sample pad by the second hydrophobic sample pad. But when the dual pad is wetted by a chase buffer that releases the specimen in whole or in part and renders the second hydrophobic sample pad permeable, the wetted specimen sample is introduced to a conjugate pad and test membrane to provide for analysis of one or more components within the specimen sample.

### FIELD OF THE INVENTION

Described herein are compositions and methods useful for preparation of fixed specimen sample fluid volume for analysis by lateral flow assays, which may be directly collected. More specifically, novel dual pad compositions and analytical devices and methods using the same for the collection of a fixed specimen volume using a dual pad comprising a first hydrophilic sample pad and a second hydrophobic pad are described. The fixed specimen volume collected on the first hydrophilic sample pad is prevented, or blocked, from saturating anything other than the hydrophilic sample pad by an abutting second hydrophobic pad. But when the sample pad is wetted by a chase buffer that releases the specimen sample in whole or in part and renders the second hydrophobic pad permeable upon diffusion of the released specimen sample into the second hydrophobic pad the wetted specimen sample may pass through the second hydrophobic pad. The dual pad composition may be provided as part of a complete lateral flow test strip, in which case a wetted specimen sample, having passed through the second hydrophobic pad may then directly proceed towards and into a conjugate pad and test membrane for analysis of one or more components within the specimen sample. Alternatively, the dual pad composition may be provided as a fixed specimen fluid volume sample collection composition for subsequent analysis by lateral flow assays. Whether provided as part of a complete lateral flow test strip or as a separate fixed specimen fluid volume sample collection composition for subsequent analysis by lateral flow assays, the dual pad composition may permit direct collection of a specimen sample without the use of intermediary capillary tubes, swabs, etc. Additionally, the dual pad compositions may provide beneficial timing flexibility as the duration that a specimen sample is held on a sample pad without further processing, or the rate at which a specimen sample wets out of a sample pad, or the rate at which a specimen sample is exposed to various blocking, amplifying, and/or cross-linking reagents can be modified by adjusting the amount of non-ionic detergent (e.g., TWEEN-20) present in the sample pad. Also described herein are analytical devices and methods specially adapted for use of the dual pad compositions described herein.

### BACKGROUND

The dual pad compositions and methods described herein address and solve problems and unmet needs associated with collection of fixed, pre-determined fluid volume samples for use with lateral flow assays. And the specimen samples may be collected directly, i.e., without the need for a capillary tube or a transfer pipet. Additionally, the dual pad compositions and methods described herein permit greater flexibility in the sampling and timing for collection of fixed, pre-determined fluid volume samples for use with lateral flow assays as specimen samples may be collected on the dual pad compositions for either immediate or subsequent further processing. Devices specially adapted for use of the dual pad compositions also facilitate and maximize the benefits achieved by the dual pad compositions and methods described herein. Accordingly, the compositions and methods provide a safer, less expensive, less wasteful, highly automatable, more precise, better controlled, and less complex alternative to traditional fixed specimen fluid volume sample collection for lateral flow assays.

### SUMMARY OF THE INVENTION

Dual pad compositions comprising a first hydrophilic sample pad and a second hydrophobic pad, and methods of using these compositions for the collection of fixed specimen fluid volume samples for subsequent lateral flow assay analyses are described. Additionally, the dual pad compositions and methods may permit the direct collection of a fluid sample, thus avoiding the need for additional fluid sample collection tubes, pipets, etc. The dual pad compositions minimally include the first hydrophilic sample pad and a second hydrophobic pad. Alternatively, the dual pad compositions may additionally comprise a conjugate pad, a test membrane, etc. After collecting a fixed specimen fluid volume sample on the first hydrophilic sample pad, a chase buffer is introduced to at least the sample pad that releases the sample from the first hydrophilic sample pad and allows it to pass or diffuse, in whole or in part, through the hydrophobic pad. The chase buffer may contain, for example, a detergent (non-ionic or otherwise). The sample pad of the dual pad composition also may be treated to provide increased timing flexibility for storage, release, and reagent modification of collected specimen samples upon introduction of chase buffer.

The dual pad compositions may be housed in various kinds of cassettes or performed as a dipstick test.

Depending on the cassette format used, the dual pad composition may be externally located on the cassette and exposed in full or in part, or it may be encased within the cassette in whole or in part provided that chase buffer can be introduced to the dual pad composition, preferably the sample hydrophilic pad, following fluid sample collection. It is also contemplated that a two-part cassette may be used that provides for sample collection on an exposed externally located dual pad composition on one part of the cassette, while a secondary part of the cassette is then added and used to cap or encase the collected sample and, optionally, to form an open window well above the collected sample for receipt of additional chase buffer.

In one preferred embodiment, adding chase buffer to cassette construction having a well-type of construction. Use of a well for receipt of added chase buffer allows the buffer to sit upon and be absorbed or wick into a previously saturated sample hydrophilic pad as the chase buffer works through, or overflows, the hydrophilic pad to render the hydrophobic pad permeable to the sample which proceeds to wick through the lateral flow assay test strip as it combines with the chase buffer.

Methods for using the dual pad compositions to collect fixed specimen fluid volume samples, either as separate fixed specimen fluid volume samples, or as part of a complete lateral flow test strip, and used, for example, as a dipstick or as part of a cassette, are described herein. Accordingly, various dual pad compositions and cassettes for housing such compositions, along with methods associated with using these various compositions are described in greater detail below.

### BRIEF DESCRIPTION OF THE DRAWINGS

FIG. 1A. FIG. 1A depicts a side view of a dual pad composition as part of a lateral flow test strip (100) comprising hydrophilic sample pad (10) and hydrophobic pad (12) with components (10) and (12) together forming the minimum dual pad composition and, also, a conjugate pad (14), a test membrane (16), and an absorbent pad handling component (22). FIG. 1B and FIG. 1C.

FIG. 1B provides an overhead view of the lateral flow test strip (100). FIG. 1C shows a three quarter aerial view of the lateral flow test strip (100). The reference identifiers used in FIG. 1B and FIG. 1C correspond to those used in FIG. 1A.

FIG. 2A. FIG. 2A provides a three quarter aerial view of the lateral flow test strip (100) held, inserted, or contained within a primary part of a two-part lateral flow assay cassette (200).

FIG. 2B. FIG. 2B is a full three quarter aerial view of the lower component (202) of a primary part of the two-part lateral flow assay cassette (200) along with the lateral flow test strip (100).

FIG. 2C. FIG. 2C is a full three quarter aerial view of two-part lateral flow assay cassette (200), wherein the primary part comprises the upper component (250) and the lower component (202) in an assembled condition and housing the lateral flow test strip (100), and further including a secondary part which is a third component separate slidable targeting cover (280).

FIG. 3A. FIG. 3A is a full three quarter aerial view of another two-part lateral flow assay cassette (300), wherein the primary part comprises the upper component (350) and the lower component (302) in an assembled condition, and wherein the secondary part comprises a third component separate rotatable targeting cover (380). Here the lateral flow assay cassette (300) is shown in an opened condition with the separate rotatable targeting cover (380) folded or rotated away from the hydrophilic sample pad (10) of the lateral flow test strip (100).

FIG. 3B. FIG. 3B is a full three quarter aerial view of the two-part lateral flow assay cassette (300). Here the lateral flow assay cassette (300) is shown in a closed condition with the separate rotatable targeting cover (380) folded or rotated down over the hydrophilic sample pad (10) of the lateral flow test strip (100).

FIG. 4. The sample pad absorption evaluation results of Example 1 are presented graphically as a bar chart in FIG. 4, wherein the volume (μL) of the whole blood uptake using a dipstick format in the configuration as generally depicted in FIG. 1 is provided for various lengths of two (2) separate sample pads. The sample pad reference numbers correspond to number 6613 (untreated conjugate pad from Ahlstrom) and 8964 (Ahlstrom), respectively. The various lengths of each of the two (2) different sample pads (A) measured 4 mm, 6 mm, and 8 mm, respectively.

### DEFINITIONS

“Blister pack” is meant to refer to a type of packaging, for example, packaging made from a semi-rigid plastic, designed to hold a single dose of a liquid, such as a chase buffer, that provides for protection and controlled dispensing of the liquid for a single use.

“Blocks” or “block” is meant to refer impeding or stopping the movement or flow of a fluid sample. As contemplated herein, the hydrophobic pad functions as an obstacle to the flow of a fluid sample from a hydrophilic pad into a hydrophobic pad unless and until chase buffer is added or introduced to the fluid sample which renders the hydrophobic pad permeable to the fluid sample.

“Cassette” is meant to refer to a device that carries or houses the dual pad composition whether it is in one or multiple parts and is understood to refer to a container, cartridge, or casing and potentially further comprising within it a series of pathways, chambers, and/or wells and, optionally, pre-loaded components such as, for example, one or more blister packs, etc. “Conjugate pad” is meant to refer to a component in a lateral flow assay that holds and releases the detection reagent, typically an antibody labeled with a visible particle (for example, gold nanoparticles), which is then mixed with the sample to detect the target molecule (for example, antigen) if present; essentially, it's the part of the lateral flow assay test strip that contains the “conjugated” antibody ready to bind to the analyte in the sample when it flows through the pad.

“Combinable” is meant to refer to something that is able to combine, unite, or merge, or that can be combined, united, or merged.

“Detergent” is meant to refer to detergent that can be used in a lateral flow assay chase or running buffer to control the flow speed, buffer the sample pH, and neutralize interferents. A well-formulated chase or running buffer can be made with a combination of components that includes not only detergent but, optionally, other various components such as, salts, surfactants, stabilizing agents, or blocking reagents, etc.

“Direct contact” is meant to refer to physical touch between two or more things, items, or components.

“Dropper” is meant to refer to a small tool used to precisely transfer small amounts of liquid by dispensing it in single drops, often used for measuring liquids where accurate dosage is important; it may consist of a small tube with a squeezable or deformable bulb or other material that, when filled with liquid, allows a user to release the liquid drop by drop.

“Exposed surface” is meant to refer to a surface that is not protected or covered, and can be touched by people or objects.

“Fixed volume” is meant to refer to a specific, unchanging amount of space occupied by a liquid substance.

“Hydrophilic” is meant to refer to the property of having a tendency to mix with, dissolve in, or be wetted by water, having an affinity for water, and capable of interacting with water through hydrogen bonding.

“Hydrophobic” is meant to refer to the property of tending to repel or fail to mix with water, wherein a hydrophobic material does not readily form hydrogen bonds with water because it is nonpolar.

“Overlap” is meant to refer to one thing or component that extends over so as to cover partly another thing or component, or to cover something partly by going over its edge.

“Protrudes” is meant to refer to a thing or component that sticks out from, or extends beyond or away from a surface.

“Reversibly impedes” refers to something that has been hindered or blocked but that be changed back to an unhindered or unblocked state.

“Rupturing” is meant to refer to the breaking, fracturing, or bursting of a thing or component such as, for example, a blister pack.

“Saturation” is meant to refer to the state or process that occurs when no more of something can be absorbed, combined with, or added to a give n thing or material such as, for example, a hydrophilic pad.

“Separable” means able to be physically separated or dissociated one thing from the other. For example, the first component and the second component as described herein, are separable.

“Test membrane” is meant to refer to the specific part of a lateral flow assay test strip where the target analyte (the substance you are testing for) binds to capture antibodies, creating a visible signal on the test strip to indicate a result; essentially, it's the “detection zone” on the lateral flow assay test strip where the test result is made apparent or read, allowing a user to see if the target molecule is present in a sample.

“Unitary” is meant to refer to a single unit or entity,

“Viewing window” is meant to refer to an opening in a wall, cover, or other structure that allows a user or viewer to see through it.

It is to be understood that any conjugations of the above defined terms shall also be understood to have the same defined meanings as appropriate in the context of the text within which such are written.

### DETAILED DESCRIPTION

Compositions for the collection of fixed specimen fluid volume samples for subsequent analysis using lateral flow assay techniques and associated cassettes and methods for using the same are described. In one preferred embodiment, the composition includes a dual pad for fixed volume direct specimen sample collection comprising a first hydrophilic sample pad and a second hydrophobic sample pad. This dual pad composition may further comprise a third conjugate pad and, optionally, a test membrane.

In one embodiment, the compositions described herein may include a dual pad for fixed volume direct specimen sample collection which comprises one or more of: a first hydrophilic sample pad having a fluid sample saturation volume of between about 5 μL to about 100 μL; a first hydrophilic sample pad that is between about 4 mm and about 8 mm in length; or a first hydrophilic sample pad that has a fluid sample saturation volume of between about 15 μL to about 35 μL.

In a preferred embodiment, the composition includes a dual pad for fixed volume direct specimen sample collection that is compatible with specimens comprising one or more of whole blood, serum, plasma, other blood component, urine, mucous, saliva, sweat, tears, and solubilized solids.

In a preferred embodiment, the composition includes a dual pad for fixed volume direct specimen sample collection, wherein a second hydrophobic sample pad blocks fluid sample flow from a first hydrophobic sample pad to the second hydrophobic sample pad. In another preferred embodiment, the composition includes a dual pad for fixed volume direct specimen sample collection, wherein the second hydrophobic sample pad reversibly impedes fluid sample flow from the first hydrophobic sample pad to the second hydrophobic sample pad.

In a preferred embodiment, the composition includes a dual pad for fixed volume direct specimen sample collection and further comprises chase buffer that is added to the dual pad and wherein the chase buffer, optionally, comprises a detergent. In certain embodiments the chase buffer wets the specimen sample collected on a first hydrophilic sample pad and a second hydrophobic sample pad.

In preferred embodiments, the composition includes a dual pad for fixed volume direct specimen sample collection, wherein a first hydrophilic sample pad and a second hydrophobic sample pad are in direct contact. In an alternative embodiment, the composition includes a dual pad for fixed volume direct specimen sample collection, wherein a first hydrophilic sample pad and a second hydrophobic sample pad overlap in part. In another preferred embodiment, the composition includes a dual pad for fixed volume direct specimen sample collection, wherein a first hydrophilic sample pad, a second hydrophobic sample pad, and a third conjugate pad are in direct contact. In another alternative embodiment, the composition includes a dual pad for fixed volume direct specimen sample collection, wherein a first hydrophilic sample pad, a second hydrophobic sample pad, and a third conjugate pad overlap in part. In another embodiment, the composition includes a dual pad for fixed volume direct specimen sample collection, wherein a first hydrophilic sample pad and a second hydrophobic sample pad are separable or combinable from a third conjugate pad.

In a preferred embodiment, a unitary cassette comprises the composition that includes a dual pad for fixed volume direct specimen sample collection. In this embodiment, the unitary cassette may include a dual pad for fixed volume direct specimen sample collection comprising a first hydrophilic sample pad and a second hydrophobic sample pad and having one or more of the following features: wherein at least the first hydrophilic sample pad of the dual pad is located on an exterior surface of the unitary cassette; wherein at least the first hydrophilic sample pad of the dual pad provides an exposed surface which protrudes outward from the unitary cassette; and wherein chase buffer is added the first hydrophilic sample pad of the dual pad by a dropper.

In another preferred embodiment, a unitary cassette comprises the composition that includes a dual pad for fixed volume direct specimen sample collection further comprises a third conjugate pad and, optionally, a test membrane.

In various embodiments of a unitary cassette comprises the composition that includes a dual pad for fixed volume direct specimen sample collection, the cassette further comprises a viewing window to observe the results shown on the test membrane.

In another preferred cassette embodiment, a two-part cassette comprises the composition that includes a dual pad for fixed volume direct specimen sample collection. In this two-part cassette embodiment, the dual pad for fixed volume direct specimen collection comprises a first hydrophilic sample pad and a second hydrophobic sample pad in a primary part. In a further embodiment of this two-part cassette, at least the first hydrophilic sample pad of the dual pad is located on an exterior surface of the primary part and, optionally, at least the first hydrophilic sample pad of the dual pad of the primary part provides an exposed surface which protrudes outward from the unitary cassette. In a further embodiment of this two-part cassette, the two-part cassette comprises an opening or internal mechanism to introduce chase buffer to the first hydrophilic sample pad of the dual pad in either the primary part or a secondary part and, optionally, the chase buffer can be added to the first hydrophilic sample pad of the dual pad via the opening by a dropper or via an internal mechanism such as a ruptured blister pack.

In another preferred cassette embodiment, a two-part cassette comprises the composition that includes a dual pad for fixed volume direct specimen sample collection. In this two-part cassette embodiment, the dual pad for fixed volume direct specimen collection comprises a first hydrophilic sample pad and a second hydrophobic sample pad in a primary part, a third conjugate pad in either the primary part or a secondary part, and a test membrane in either the primary part or a secondary part.

In various embodiments of the two-part cassette, said cassette may further comprise a viewing window to observe the results shown on the test membrane in either the primary part or a secondary part.

In a preferred embodiment, the compositions described herein support methods of for direct collection of a fixed specimen volume comprising the direct introduction of a specimen to a dual pad comprising a first hydrophilic sample pad and a second hydrophobic sample pad. In various embodiments, methods comprise introduction of a specimen and using a unitary cassette or a two-part cassette. In various embodiments, methods comprise use of a dual pad comprising a first hydrophilic sample pad and a second hydrophobic sample pad for analysis of a directly collected fixed specimen volume.

In a preferred embodiment compositions including a dual pad comprising a first hydrophilic sample pad and a second hydrophobic sample pad may be provided as part of a kit further comprising at least one of a separable chase buffer container and instructions for use.

In another preferred embodiment compositions including a dual pad comprising a first hydrophilic sample pad and a second hydrophobic sample pad may be provided as part of a kit further comprising at least one of a cassette comprising a blister pack and instructions for use.

### Fixed Specimen Fluid Volume Sample Compositions

Fixed specimen fluid volume samples are collected using dual pad compositions, comprising a first hydrophilic sample pad and a second hydrophobic pad. The first hydrophilic sample pad may be constructed and sized to allow saturation by an introduced fluid sample having a certain predetermined value, typically measured in (μL). The second hydrophobic sample pad reversibly impedes fluid sample flow from the first hydrophobic sample pad to the second hydrophobic sample pad. That is, when a chase buffer comprising detergent is introduced to the first hydrophobic sample pad, the second hydrophobic sample pad is rendered permeable and the wetted specimen sample together with the chase buffer can proceed through the second hydrophobic sample pad (which is no longer hydrophobic in the presence of the chase buffer).

In preferred embodiments, for example, fluid samples having volume anywhere between about 5 μL to about 100 μL may be collected in fixed predetermined amounts. That is, fluid volumes of about 5 μL, about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, and about 100 μL may be collected using the dual pad compositions described herein.

In another embodiment, such as with a very thin dipstick, fluid samples having volume anywhere between about 2 μL to about 3 μL, may be may be collected using the dual pad compositions described herein. It is contemplated that fluid samples having volumes greater than 100 μL, while they may be used, might require an undesirably long assay run time. In some embodiments, a standard deviation in the sample volume collected of about 1-3 μL may be acceptable.

Specimen fluid samples may comprise any one or more of whole blood, serum, plasma, other blood component, urine, mucous, saliva, sweat, tears, and solubilized solids.

The first hydrophilic sample pad may comprise typical sample pads and conjugate pads used in the lateral flow industry, including without limitation, for example, 8964, 1660, 1662, 1663 and treated 6613 pads (Ahlstrom), VF2, CF1, and CF3 (Cytiva). The size of the hydrophilic sample pad may vary depending on the targeted or predetermined fixed volume and/or sample type that it is intended to collect. In a preferred embodiment, typical lengths may range from about 2-20 mm and typical widths may range from about 1-8 mm to control the volume of sample that is acquired.

The second hydrophobic pad may comprise any untreated hydrophobic pad that is typically used in lateral flow immunoassays. This pad material may be polyester in nature or another hydrophobic material and may include, for example, pads such as 6613 and 6615 (Ahlstrom). The size of the hydrophobic pad may vary depending upon the delay time that is required for the assay under question. Delay times may also be made to vary, or controlled, by the surfactant concentration present in the buffer. In an embodiment, delay times may be from 0 seconds and up to or equal to about one of 15 seconds, 30 seconds, 45 seconds, 1 minute, 1 minute and 15 seconds, 1 minute and 30 seconds, 1 minute and 45 seconds, 2 minutes, 2 minutes and 15 seconds, 2 minutes and 30 seconds, 2 minutes and 45 seconds, 3 minutes, 3 minutes and 15 seconds, 3 minutes and 30 seconds, 3 minutes and 45 seconds, 4 minutes, 4 minutes and 15 seconds, 4 minutes and 30 seconds, 4 minutes and 45 seconds, or 5 minutes. In a preferred embodiment, the delay times may range from about 15 seconds to about 3 minutes.

In a preferred embodiment, typical lengths may range from about 4-16 mm and typical widths may range from about 1-8 mm to match the sample pad width.

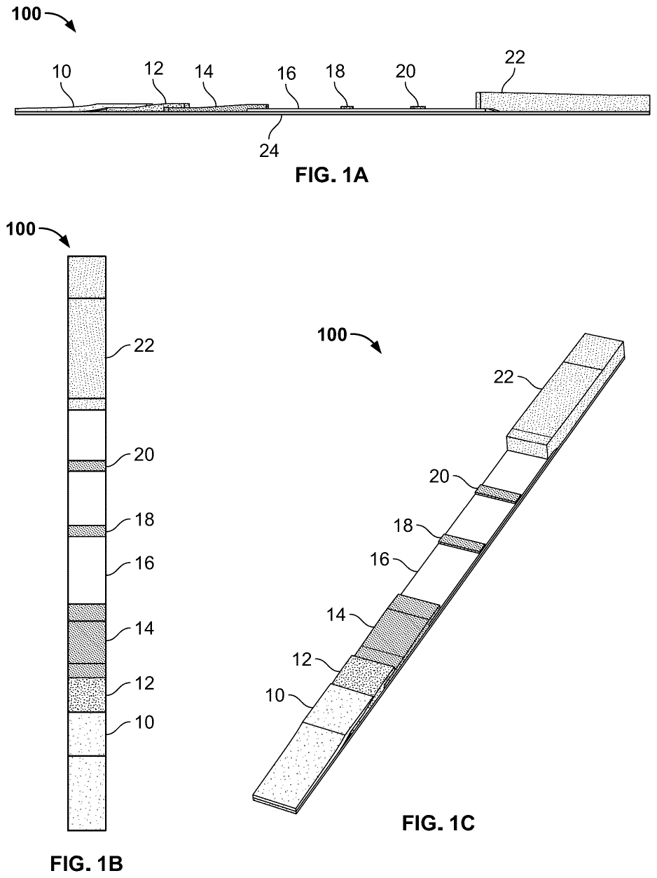

The first hydrophilic sample pad and the second hydrophobic pad are in contact with each other. In a preferred embodiment, the first hydrophilic sample pad and the second hydrophobic pad are in direct contact with each other, wherein an edge of the hydrophilic sample pad is abutted by an edge of the hydrophobic pad. In another preferred embodiment, the first hydrophilic sample pad and the second hydrophobic pad are positioned as depicted in FIG. 1A, such that there is a slight overly by the first hydrophilic sample pad over the second hydrophobic pad. This embodiment is preferred because the chase buffer is typically added to the hydrophilic sample pad. The detergent present in the chase buffer quickly diffuses to the hydrophobic pad and will wet out the pad. This embodiment allows the end user to add specimen sample and chase buffer to the same location, i.e., to the hydrophilic sample pad. After the hydrophobic pad is wetted out, the sample may migrate freely through the pad and into the conjugate pad via capillary action.

The first hydrophilic sample pad and the second hydrophobic pad are connected to a support base, or backing card. In a preferred embodiment the support base, or backing card, has a pressure sensitive adhesive layer so that each of the hydrophilic sample pad, the hydrophobic pad, the conjugate pad, and the test membrane may be directly applied or adhered to the support base, or backing card.

The dual pad compositions may additionally comprise one or more of a conjugate pad, a test membrane, a holding component, and a support base (e.g., a backing card with pressure-sensitive adhesive). Additional components that may be included with the dual pad compositions may include conventional lateral flow assay conjugate pad and/or test membrane technology.

### Cassettes

Various cassette formats may be used with the dual pad compositions described herein. In a preferred cassette, the dual pad composition or only the hydrophilic sample pad of the dual pad composition may be externally located on the cassette, in full or in part, and exposed, in full or in part, or it may be encased within the cassette, in whole or in part, provided that each of the fluid specimen sample and the chase buffer can be introduced to the dual pad composition or only the hydrophilic sample pad of the dual pad composition.

In a preferred embodiment, a unitary cassette may be used which does not include a secondary part targeting cover. In such an embodiment, chase buffer may be introduced directly to the hydrophilic sample pad of the dual pad composition by using a dropper or other mechanical distribution device, or by dipping the hydrophilic pad comprising a specimen sample into a vial or other container comprising buffer fluid.

In a preferred embodiment, a two-part cassette may be used that provides for specimen sample collection on an exposed externally located hydrophilic sample pad of the dual pad composition on a primary part of the cassette. The specimen sample may be collected directly by touching the exposed externally located hydrophilic sample pad to the specimen sample. Subsequently, a secondary part of the cassette, a targeting cover, which functions, in part, as a protective cap, is then added, moved into place, and combined with the primary part of the cassette and used to encase the collected specimen sample on the hydrophilic sample pad which becomes only partially exposed and internally located, but was previously fully exposed and externally located. The secondary part of the cassette, a targeting cover, can also function as a chase buffer targeting structure by forming an open window well directly above the collected specimen sample on the hydrophilic sample pad for the introduction of additional chase buffer once it is added, moved into place, and combined with the primary part of the cassette. In an embodiment, the open window well may be directly above and in physical contact with the collected specimen sample on the hydrophilic sample pad for the introduction of additional chase buffer.

In an embodiment, the two-part cassette comprises two parts that are completely separable and, optionally, wherein each of the two parts includes adaptations so that the two parts snap, click, lock, or fit, releasably or permanently, together. In another embodiment, the two-part cassette comprises two parts that are connected as one unit and wherein each of the two parts is movable in relationship to the other such as, for example, by a hinged connection, and each of the two parts includes adaptations so that the two parts snap, click, lock, or fit, releasably or permanently, together. In an embodiment, the primary part of the two-part cassette comprises an upper component and a lower component as a fixed and combined part of the two-part cassette and, optionally, the upper component of the primary part comprises a viewing window so that assay results can be observed.

In still another embodiment, a two-part cassette may be used that provides for sample collection on an exposed externally located dual pad composition on a primary part of the cassette, while a secondary part of the cassette is then added or combined with the primary part of the cassette and used to enclose or encase, partially or completely, the collected sample on the hydrophilic sample pad.

In an embodiment, for any two-part cassette, the chase buffer may be introduced to the hydrophilic sample pad of the dual pad composition via an open chase buffer introduction hole located in the targeting cover.

In another embodiment, for any two-part cassette, the chase buffer may be introduced to the hydrophilic sample pad of the dual pad composition by bursting a blister pack containing the chase buffer onto the hydrophilic sample pad. In this other embodiment, the blister pack may be housed in either the primary part of the cassette which carries the dual pad composition or the secondary part of the cassette which covers, in whole or in part, the dual pad composition. It is contemplated that the release mechanism for the blister pack may be an external push button located on the primary part or secondary part of the cassette. Alternatively, the release mechanism for the blister pack may be automatically triggered by the physical act of combining the primary part of the cassette with the secondary part of the cassette. In such embodiments, the secondary part of the cassette does not require inclusion of via an open chase buffer introduction hole located in the targeting cover.

FIG. 1A, Figure, 1B, and FIG. 1C provide a non-limiting example of a lateral flow test strip suitable for use with cassettes contemplated herein. As shown, FIG. 1A depicts a side view of a dual pad composition as part of a lateral flow test strip (100) comprising hydrophilic sample pad (10) and hydrophobic pad (12) with components (10) and (12) together forming the minimum dual pad composition and, also, a conjugate pad (14), a test membrane (16), and an absorbent pad handling component (22).

Each of sample pad (10, hydrophobic pad (12), conjugate pad (14), test membrane (16), and handling component (22) are shown on top of a supportive component (24) which spans underneath the components and serves as a base to which the components are connected, attached, and/or adhered. As shown, a test line (18) and control line (20) are also indicated. Sample pad (10) may include additional features and activities such as blocking reagents, agglutination reagents (i.e., to hold whole blood) or lysing reagents (e.g., to release target that is within a virus or bacteria). To be clear, the lateral flow test strip (100) may be used as a dipstick. Also, a variation on the lateral flow test strip (100) not shown, contemplates a similar but abbreviated structure wherein the hydrophilic sample pad (10) and hydrophobic pad (12) along with a supportive component (24) are provided as a separate and independent composition for the preparation and/or collection of fixed specimen fluid volume samples, wherein said collection may be direct collection.

In use, a fluid sample is applied to the hydrophilic sample pad (10). A wide variety of hydrophilic sample pad (10) can be used. In a preferred embodiment due to demonstrating the most uniform uptake of sample volume on testing, VF2 and ‘8964’ are used as hydrophilic sample pad (10). The capillary action of the fluid sample through the pad is halted when the specimen sample reaches the hydrophobic pad (12); thus, limiting the volume of specimen sample that is collected to a fixed amount that may be absorbed by the sample pad (10). A wide variety of hydrophobic pad (12) can be used. In one embodiment, a polyester based pad is used such as, for example, an untreated ‘6613’ conjugate pad from Ahlstrom. It is contemplated that other polyester based pads would also work, for example, a ‘6615’ pad.

After application of a wetting reagent (e.g., a chase buffer containing detergent) (not shown), most preferably to the hydrophilic sample pad (10), the wetted sample can then proceed via capillary action through the hydrophobic pad (12) towards a conjugate pad (14) and a test membrane (16). Once the chase buffer containing detergent is added, the specimen sample is ‘pushed’ up as the TWEEN-20 diffuses to hydrophobic pad (12). In a less preferred embodiment, the wetting reagent can be added to hydrophobic pad (12); however, introducing the wetting reagent to hydrophobic pad (12) rather than hydrophilic sample pad (10) is expected to be less efficient.

Once the wetted sample arrives at, and is introduced to, the conjugate pad (14), the lateral flow test strip (100) may proceed according to conventional standards. That is, the wetted sample will flow onto the conjugate pad (14) containing conjugated nanoparticles that interact with the analyte of interest in the sample. The reaction volume then proceeds up the test membrane (16) where a test line (18) and control line (20) are striped. If the sample contains the analyte of interest, a test line and control line will appear on the membrane. If the analyte is not present in the sample, then only the control line will appear. The entire reaction volume is absorbed (via capillary action) into the absorbent pad handling component (22).

FIG. 1B provides an overhead view of the lateral flow test strip (100) and FIG. 1C shows a three quarter aerial view of the lateral flow test strip (100). The reference identifiers used in FIG. 1B and FIG. 1C correspond to those used in FIG. 1A.

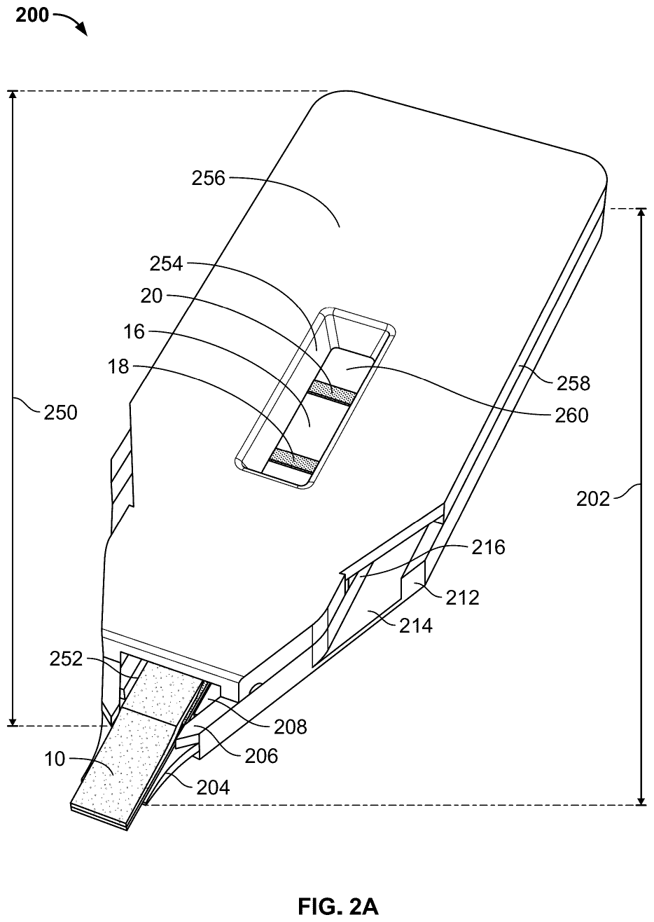

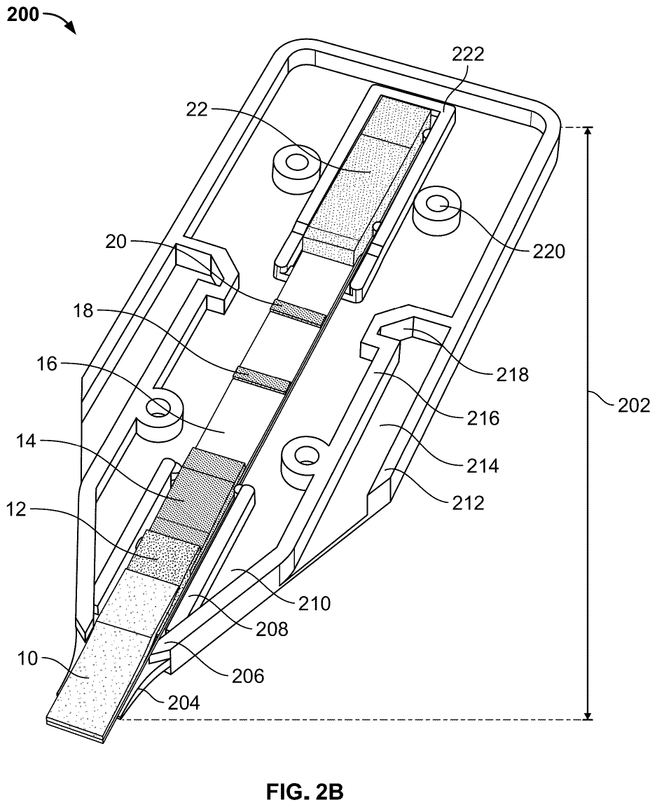

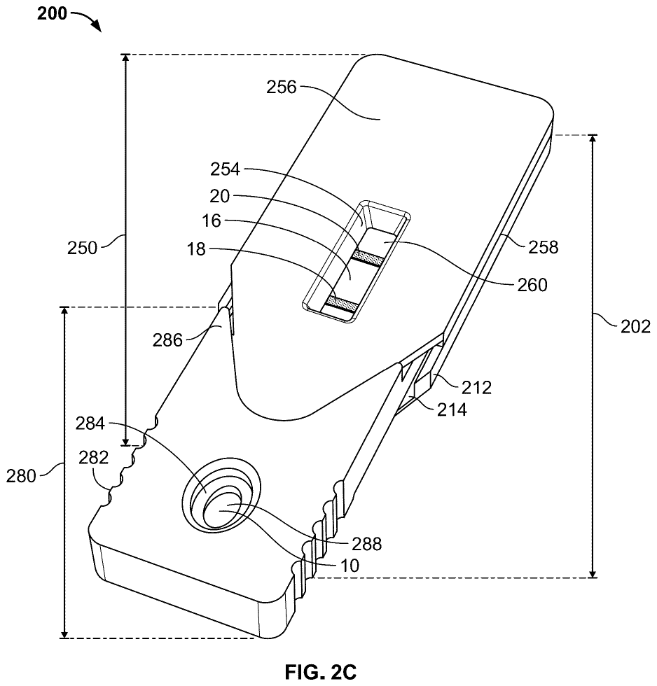

FIG. 2A, FIG. 2B, and FIG. 2C provide an example two-part cassette wherein the primary part of the cassette and the secondary part of the cassette are completely separable. FIG. 2A provides a three quarter aerial view of the lateral flow test strip held, inserted, or contained within a primary part of a two-part lateral flow assay cassette (200). As shown the lateral flow assay cassette (200) comprises a primary part including two primary components, lower component (202) and upper component (250). From the image it can be seen that the lateral flow test strip is visible in part, with the hydrophilic sample pad (10) extending outside of the lateral flow assay cassette (200). Also visible, due to the presence of viewing window (254) in the upper component (250) is the test membrane (16) where a test line (18) and control line (20) are striped.

The lower component (202) forms the lower part of the lateral flow assay cassette (200). As shown, lower component (202) comprises a hydrophilic sample pad (10) landing area (204) which extends away from the main body of the lateral flow assay cassette (200). The lower component (202) also comprises placement prongs (206) and internal guide walls (208) to hold the lateral flow test strip (100) in proper position. The lower component (202) also comprises a receiving alley (214) for physical slidable connection with a slidable targeting cover (280) (not shown in FIG. 2A) as shown in FIG. 2C. The lower component (202) further comprises lower component exterior walls (212) and interior walls (216).

The upper component (250) forms the upper part of the lateral flow assay cassette (200). As shown, upper component (250) comprises viewing window (254), lateral flow test strip opening (260), top planar surface (256), upper component exterior walls (258), and a lateral flow test strip (100) opening or orifice (252).

FIG. 2B is a full three quarter aerial view of the lower component (202) of a primary part of the two-part lateral flow assay cassette (200). All of the features referenced in FIG. 2A in connection with lower component (202) are included again in FIG. 2B. Additionally, also shown in FIG. 2B is hook area (218) which provides a physical attachment point for arms (not shown) of slidable targeting cover (280) (not shown in FIG. 2A) as shown in FIG. 2C. Additionally depicted are attachment point(s) (220) which may be used to connect the lower component (202) with upper component (250). Further depicted is bookend structure (222) which holds the lateral flow test strip (100) in proper position.

FIG. 2C is a full three quarter aerial view of two-part lateral flow assay cassette (200), wherein the primary part comprises the upper component (250) and the lower component (202) in an assembled condition, and further including a secondary part which is a third component separate slidable targeting cover (280). The slidable targeting cover (280) is shown in slid into place, or in combination with, the assembled or combined upper component (250) and the lower component (202). Not visible here are the arms of the slidable targeting cover (280) which extend into the receiving alley (214) formed by interior walls (216) in the lower component (202). Also not visible here are the prongs located at the end of the arms of the slidable targeting cover (280) which may snap into place with one or more hook area (218) formed in the lower component (202). The form and function of the slidable entry and physical attachment point for arms (not shown) of slidable targeting cover (280) can be appreciated by reference to FIG. 2B which depicts the internal construction of the lower component (202) and this FIG. 2C which shows the upper component (250) and the lower component (202) in an assembled condition and in combination with the slidable targeting cover (280) slid into place of the lateral flow assay cassette (200).

All of the features referenced in FIG. 2A and FIG. 2B are included again in FIG. 2C. Additionally, also shown in FIG. 2C is the slidable targeting cover (280). The slidable targeting cover (280) is shown with grip zone (282) for ease of handling the slidable targeting cover (280). An open bottomed well (284) is also shown along with the open chase buffer introduction hole (288). The slidable targeting cover (280) interface (286) provides a contact surface between the slidable targeting cover (280) and the upper component (250) when the slidable targeting cover (280) is fully slid into place.

Also included in FIG. 2C is a lateral flow test strip (100) shown inside the fully assembled and combined lateral flow assay cassette (200), comprising each of the upper component (250), the lower component (202), and the slidable targeting cover (280). Also visible, due to the presence of the viewing window (254) and the lateral flow test strip opening (260) in the upper component (250), is the test membrane (16) where a test line (18) and control line (20) are striped. Also visible, due to the presence of the open bottomed well walls (284) and open chase buffer introduction hole (288) is the sample pad (10) of the lateral flow test strip (100). In use, the open bottomed well walls (284) and chase buffer introduction hole (288) can be used to target and receive chase buffer introduced into the chase buffer introduction hole (288) and, thus, into contact with the sample pad (10) of the lateral flow test strip (100).

Also not visible here is the interior or the bottom surface of the slidable targeting cover (280). The slidable targeting cover (280), in a preferred embodiment, includes a recessed pocket to receive the lateral flow test strip (100). The slidable targeting cover (280) serves to protect the specimen sample and lateral flow test strip (100) by encasing the same on at least three sides in a rigid or semi-rigid slidable targeting cover (280). Alternatively, the slidable targeting cover (280) may serves to protect the specimen sample and lateral flow test strip (100) by encasing the same on just two sides in a rigid or semi-rigid slidable targeting cover (280) while the landing area (204) of lower component (202) which extends away from the main body of the lateral flow assay cassette (200) provides support and protection for the bottom surface of the lateral flow test strip (100). Meanwhile, the targeting cover also comprises open bottomed well walls (284) and chase buffer introduction hole (288) in order to facilitate and target the introduction of chase buffer to the appropriate location on the lateral flow test strip (100), the sample pad (10).

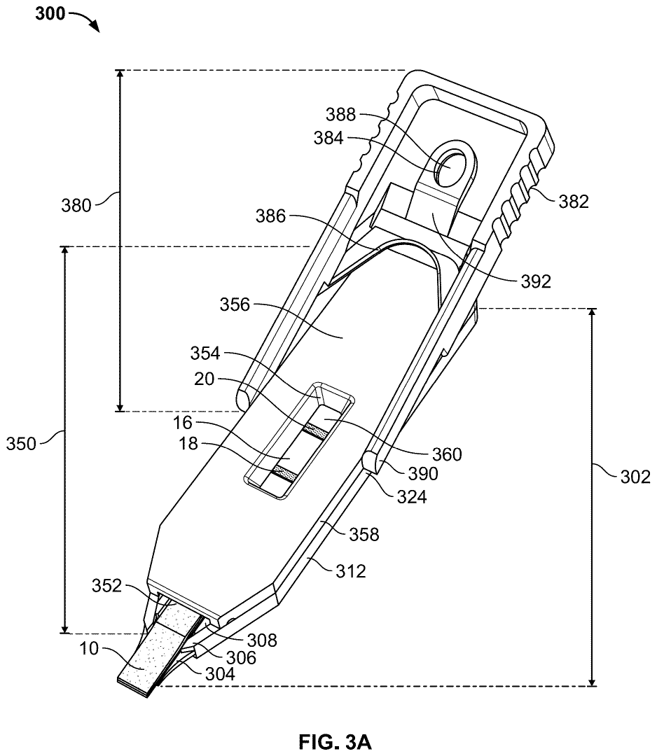

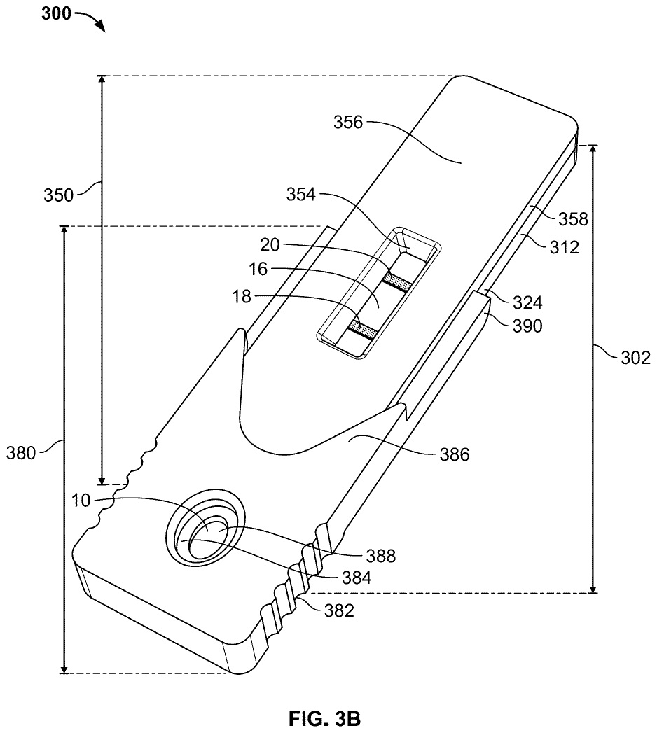

FIG. 3A is a full three quarter aerial view of another two-part lateral flow assay cassette (300), wherein the primary part comprises the upper component (350) and the lower component (302) in an assembled condition, and wherein the secondary part comprises a third component separate rotatable targeting cover (380). Here the lateral flow assay cassette (300) is shown in an opened condition with the separate rotatable targeting cover (380) folded or rotated away from the hydrophilic sample pad (10) of the lateral flow test strip (100).

Similarities in the design between lateral flow assay cassette (200) and lateral flow assay cassette (300) can be seen, however, a major change to the lateral flow assay cassette (300) is how the rotatable targeting cover (380) attaches or combines with the upper component (350) and the lower component (302). In lateral flow assay cassette (300) the rotatable targeting cover (380) attaches to the upper component (350) and the lower component (302) in a hinged manner which allows it to be rotated into position over the lateral flow test strip (100). Also, the upper component (350) and the lower component (302) are simplified. For example, the lower component (302) does not include the receiving alley (214) or a hook area (218) as formed by interior walls (216) in the lower component (202). As shown, the rotatable targeting cover (380) attaches at rotatable hinge (390) to one or more exterior side walls (312) of the lower component (302) at one or more attachment points (324).

The lower component (302) further comprises a hydrophilic sample pad (10) landing area (304) which extends away from the main body of the lateral flow assay cassette (300) when in an opened condition. The lower component (302) also comprises placement prongs (306) and internal guide walls (308) to hold the lateral flow test strip (100) in proper position.

The upper component (350) forms the upper part of the lateral flow assay cassette (300). As shown, upper component (350) comprises viewing window (354), lateral flow test strip opening (360), top planar surface (356), upper component exterior walls (358), and a lateral flow test strip (100) opening or orifice (352). Also visible, due to the presence of the viewing window (354) and the lateral flow test strip opening (360) in the upper component (350), is the test membrane (16) where a test line (18) and control line (20) are striped.

The rotatable targeting cover (380) comprises is shown with grip zone (382) for ease of handling the rotatable targeting cover (380). An open bottomed well (384) is also shown along with the open chase buffer introduction hole (388). The rotatable targeting cover (380) interface (386) provides a contact surface between the rotatable targeting cover (280) and the upper surface (356) of the upper component (350) when the rotatable targeting cover (280) is fully rotated into place. Ramp (392) further encloses the opening or orifice (352) and bends towards the lateral flow test strip (100) when the rotatable targeting cover (380) is moved from an open condition as shown in FIG. 3A to closed condition as shown in FIG. 3B.

FIG. 3B is full three quarter aerial view of the two-part lateral flow assay cassette (300) with the primary part comprising the upper component (350) and the lower component (302) in an assembled condition and further including a secondary part third component separate rotatable targeting cover (380). Here the lateral flow assay cassette (300) is shown in a closed condition with the separate rotatable targeting cover (380) folded or rotated down over the hydrophilic sample pad (10) of the lateral flow test strip (100).

All of the features referenced in FIG. 3A are included again in FIG. 3B. Here it is shown that The rotatable targeting cover (380) comprises is shown with grip zone (382) for ease of handling the rotatable targeting cover (380). An open bottomed well (384) is also shown along with the open chase buffer introduction hole (388). The rotatable targeting cover (380) interface (386) provides a contact surface between the rotatable targeting cover (280) and the upper surface (356) of the upper component (350) when the rotatable targeting cover (280) is fully rotated into place. Ramp (392) (not visible) further encloses the opening or orifice (352) (not visible) and bends towards the lateral flow test strip (100) when the rotatable targeting cover (380) is in the closed condition as shown in FIG. 3B.

The specific cassette formats described herein are non-limiting. In general, cassettes of various formats may be used; however, in each instance where a conventional lateral assay flow cassette is contemplated, specific adaptations and adjustments will be needed in order to render such cassette designs suitable for use with the fixed specimen fluid volume sample dual pad compositions described herein.

### Methods

Methods for using the compositions described herein for the collection of fixed specimen fluid volume samples and for subsequent lateral flow assay analyses are described.

A specimen sample may be collected by touching or contacting the hydrophilic sample pad directly to or with the specimen sample. In a preferred embodiment, the specimen sample is directly collected with a specimen sample. For example, the hydrophilic sample pad of the dual pad composition is placed in direct contact with the specimen sample, such as, for example, when the hydrophilic sample pad of the dual pad composition is placed in direct contact with a bleeding tissue surface or pricked finger, or directly on the surface of a test subject's tongue, or directly against a mucous membrane. In another embodiment, the sample may be collected indirectly, for example by using a test tube, capillary tube, etc.

In a preferred embodiment, the hydrophilic sample pad is exposed to a specimen sample until it is completely saturated with the sample. Saturation may be determined visually, by following prescribed exposure times (such as, for example, about five (5) seconds, or about ten (10) seconds, or about fifteen (15) seconds), or when it is otherwise determined that no additional specimen sample is wicking into the hydrophilic sample pad. The size and the carrying capacity of the hydrophilic sample pad, bordered by the hydrophobic pad, will determine the amount of the fixed specimen fluid volume sample absorbed into the dual pad composition.

In one embodiment, the hydrophilic sample pad may collect a specimen sample by touching the hydrophilic sample pad directly to the specimen sample until the hydrophilic sample pad is saturated. In one embodiment, the hydrophilic sample pad is held or housed in a primary part of a two-part cassette wherein the hydrophilic sample pad extends away from the main body of the lateral flow assay cassette and is touched directly to the specimen sample.

In another embodiment, the hydrophilic sample pad may collect a specimen sample by using an intermediary specimen sample collection device, such as a transfer pipet, capillary tube, etc. that can be used to introduce specimen sample to the hydrophilic sample pad.

Chase buffer suitable for use with the dual pad compositions described herein may include a detergent (non-ionic or otherwise). In one embodiment, a chase buffer containing detergent (e.g., about 0.01%-2% TWEEN-20 or about 0.01%-2% Triton X-100) may be added to the hydrophilic sample pad. This may be done by placing a dipstick comprising the dual pad composition (including the hydrophilic pad) along with the other components of a lateral flow test strip into a test or holder well that contains the chase buffer, or by simply adding the chase buffer to the hydrophilic sample pad part of the dipstick using a dropper or other mechanism.

In an alternative embodiment, the dual pad compositions including other components of a lateral flow test strip, e.g., a conjugate pad, test membrane, and absorbent pad, may be assembled into a cassette and the chase buffer may then be added to the hydrophilic sample pad. When the chase buffer and detergent flow into the hydrophilic sample pad (which comprises a specimen sample for conducting a lateral flow test) and interface with the hydrophobic pad, the hydrophobic pad will start to “wet out” in the presence of the detergent. In this manner, the lateral flow test reaction will then proceed as normal. The collected specimen sample will flow through the conjugate pad, releasing labeled particles into the membrane and binding to the test line if the specific reaction has occurred.

The detergent type and concentrations and chase buffer amount volumes used to make the hydrophilic sample pads wettable may vary.

Generally, the lateral flow tests may comprise the dual pad compositions in either a dipstick or in a cassette format. The lateral flow tests may be performed horizontally or vertically. The length of the hydrophilic sample pad used in the lateral flow tests may vary. The length of the hydrophobic pads used in the lateral flow tests may vary. In preferred embodiments, the hydrophilic sample pad may be of a length equal to or about any one of 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, or 60 mm. One method is to use the dual pad composition together with the conjugate pad, test membrane, and absorbent pad to form a dipstick assay. In this method, a specimen sample is collected in a fixed volume using the dual pad compositions described herein. A chase buffer is then introduced to the hydrophilic sample pad. The chase buffer (containing detergent) will then wet out the hydrophobic pad, causing the specimen sample to travel through the hydrophobic pad and to the conjugate pad and test membrane, and then to the absorbent pad. Typical volumes of chase buffer may range from about 30 μL to about 300 μL, although about 90 μL (approximately 3 drops) may be ideal for this application.

Another method involves using a cassette. For example, the two-part cassette embodiments described above may be used.

In one embodiment, the two-part cassette described in FIG. 2A, FIG. 2B, and FIG. 2C can be used. The primary part of the two-part cassette comprises a lower component and an upper component combined together to form a single unit. A lateral flow test strip is partially housed within the primary part; however, the hydrophilic sample pad of the dual pad assembly (comprising both the hydrophilic sample pad and the hydrophobic sample pad) extends outside of the primary part of the lateral flow assay cassette with its hydrophilic surface exposed. Specifically, a lower component which forms the lower part of the primary part of the lateral flow assay cassette includes a hydrophilic sample pad landing area which extends away from the main body of the primary part of lateral flow assay cassette and supports the exposed hydrophilic pad, in full or in part, from underneath or on a side opposite to the exposed hydrophilic surface of the hydrophilic pad. The lower component of the primary part of the cassette also comprises placement prongs and internal guide walls to assist placement of and hold the lateral flow test strip in proper position.

After a specimen sample is collected on the exposed surface of the hydrophilic pad which extends away from the main body of the primary part of cassette, a secondary part slidable targeting cover is placed in connection, or combined, with the primary part of the two-part assay to form a complete combined two-part assay device. The second slidable targeting cover is specially adapted for combination with the primary part of the cassette. Accordingly, the secondary part slidable targeting cover includes arms which insert into and are received by one or more receiving alleys in the primary part of the cassette. The arms of the secondary part slidable targeting cover may include prongs or extensions that hook into hook areas preformed and provided as part of the primary part of the cassette. Once the secondary part slidable targeting cover is fully inserted into the primary part of the cassette, the two components may be permanently connected.

Once the primary part and the secondary part of the cassettes are fully assembled around the lateral flow assay strip, the chase buffer may be introduced to the hydrophilic sample pad. The chase buffer can be introduced to the hydrophilic sample pad via a window or access point provided in the top surface of the secondary part slidable targeting cover. Also visible in the fully assembled cassette which includes the lateral flow assay strip are the test membrane where a test line and control line are striped which can be seen or observed using the results viewing window provided in the primary part upper component of the cassette.

In another cassette embodiment, as depicted in FIG. 3A and FIG. 3B, the methods for using the cassette may be similar to that described above in connection with sample collection using the primary part of the two-part cassette. However, the secondary part of the two-part cassette differs in how it connects to the primary part of the two-part cassette. Specifically, the secondary part of the cassette may be permanently attached to the primary part of the cassette via a hinge mechanism that allows the secondary part to be rotated into place after a specimen sample has been collected. Once the secondary part rotatable targeting cover of the two-part cassette is rotated into place, the chase buffer may be introduced to the hydrophilic sample pad. The chase buffer can be introduced to the hydrophilic sample pad via a window or access point provided in the top surface of the secondary part rotatable targeting cover.

Additional methods for using the dual pad compositions described herein may involve cassettes which include an internal blister pack comprising chase buffer. In an embodiment, instead of adding chase buffer via an external approach or mechanism such as, for example, a dropper, an internal blister pack can be ruptured to introduce chase buffer to the hydrophilic pad saturated with specimen sample. In certain embodiments, the blister pack can be ruptured by a press button located on an external surface of the cassette, either part one or part two. In other embodiments, the blister pack can be automated such that it is ruptured mechanically upon combination of the primary part and the secondary part of a two-part cassette. For example, the internal blister pack may be positioned such that it is poked by a pin or sufficiently sharp point upon the combination of the primary part and the secondary part of a two-part cassette.

The above compositions, cassettes, and methods are not intended to be limiting. It is contemplated that various additional compositions, cassettes, and methods comprising the dual pad compositions described herein may be developed and used.

### EXAMPLES

### 1. Example 1: Sample Pad Evaluation

Two different hydrophilic sample pads (“treated 6613” and “8964”) were evaluated for fixed specimen sample fluid volume accuracy using different lengths of hydrophilic sample pad together with other lateral flow test components in the configuration shown in FIG. 1A, FIG. 1B, and FIG. 1C. The sample pad numbers correspond to number 6613 (treated conjugate pad from Ahlstrom) and 8964 (Ahlstrom), respectively. The 6613 pad was pre-treated with surfactant (in this example, 0.5% TWEEN-20) for wettability as, otherwise, its polyester is hydrophobic.

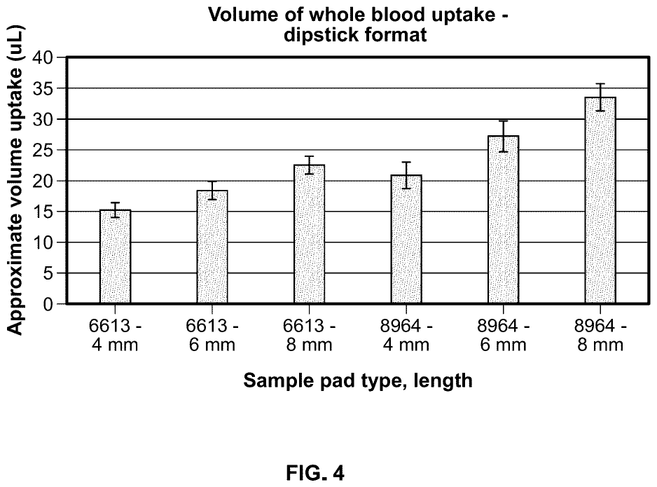

Dual pad compositions comprising the hydrophilic sample pads (“treated 6613” and “8964”) were tested in a dipstick format, wherein specimen sample was introduced to and saturated the hydrophilic pads which were provided as part of a dual pad composition as described herein. Measurements were acquired using an analytical balance. Briefly, a volume of whole blood sample was loaded onto parafilm located in the analytical balance. The mass of the whole blood was recorded. The hydrophilic end of the dipstick was then touched against the whole blood until the hydrophilic sample pad was completely wetted out. No additional sample volume could enter the test as the second hydrophobic pad prevented any further flow. The mass shown on the analytical balance was then recorded and the difference in mass (before and after introduction of the dipstick) was used to determine the volume absorbed by the dipstick hydrophobic pad. The dipsticks were held touching the edge of the hydrophilic sample pad directly to a specimen sample of whole blood for 10 seconds to ensure complete saturation of the hydrophilic sample pad. The results of this evaluation are provided in Table 1, and presented graphically in FIG. 4.

| Sample hydrophilic sample | Average μL (n = | Standard |
|---|---|---|
| pad type - length | 5 replicates | deviation μL |
| Treated 6613 - 4 mm | 15.2 | 1.20 |
| Treated 6613 - 6 mm | 18.38 | 1.49 |
| Treated 6613 - 8 mm | 22.52 | 1.44 |
| 8964 - 4 mm | 20.88 | 2.13 |
| 8964 - 6 mm | 27.18 | 2.54 |
| 8964 - 8 mm | 33.52 | 2.19 |

Note:
all 6613 pads were treated with 0.5% TWEEN-20

The hydrophilic sample pad absorption evaluation results of Example 1 are presented graphically as a bar chart in FIG. 4, wherein the volume (μL) of the whole blood uptake is provided for various lengths of the two different hydrophilic sample pads (“treated 6613” and “8964”). The various lengths of each of the two (2) different sample pads measured 4 mm, 6 mm, and 8 mm, respectively. The “treated” pads may be treated with a buffer containing 0.1%, 0.5% or 1% TWEEN-20 or similar non-ionic detergent. Here, all treated sample pads were treated with 0.5% TWEEN-20.

Each of the two different hydrophilic sample pads (“treated 6613” and “8964”) is at the end of a dipstick, as shown in FIG. 1A, FIG. 1B, and FIG. 1C. The length of this hydrophilic sample pad is altered based upon the desired volume to be collected. The next component that is attached, connected, adhered, or laminated in the lateral flow test strip is a hydrophobic pad (e.g., an untreated “6613” pad) indicated with reference identifier (12) in FIG. 1A, FIG. 1B, and FIG. 1C. To be clear, the treated 6613 pads are “pre-treated” so that these sample pads are hydrophilic versions of the typically hydrophobic 6613 pad (polyester base).

When biological fluid (e.g., serum, whole blood, urine, etc.) touches the bottom of the hydrophilic sample pad, it is quickly wicked into the entire hydrophilic sample pad via capillary action. However, after reaching the end of the hydrophilic sample pad, the capillary action stops once the hydrophobic barrier of the hydrophobic pad is reached. In this way, a controlled and fixed volume of biological specimen may be acquired. As shown by the results of Example 1, various types of hydrophilic sample pads, cut to various sizes, can be used to reliably, accurately, and reproducibly provide for preparation of fixed specimen sample fluid volume for analysis by lateral flow assays, which may be directly collected.

1. A dual pad for fixed volume direct specimen sample collection comprising a first hydrophilic sample pad and a second hydrophobic sample pad.

2. The dual pad of claim 1, wherein said dual pad further comprises a third conjugate pad.

3. The dual pad of claim 1, wherein said dual pad further comprises a test membrane.

4. The dual pad of claim 1, wherein said first hydrophilic sample pad has a fluid sample saturation volume of between about 5 μL to about 100 μL.

5. The dual pad of claim 1, wherein the first hydrophilic sample pad is between about 4 mm and about 8 mm in length.

6. The dual pad of claim 1, wherein the specimen comprises one or more of whole blood, serum, plasma, other blood component, urine, mucous, saliva, sweat, tears, and solubilized solids.

7. The dual pad of claim 1, wherein the second hydrophobic sample pad blocks fluid sample flow from the first hydrophobic sample pad to the second hydrophobic sample pad.

8. The dual pad of claim 1, wherein the second hydrophobic sample pad reversibly impedes fluid sample flow from the first hydrophobic sample pad to the second hydrophobic sample pad.

9. The dual pad of claim 1, further comprising added chase buffer and wherein the added chase buffer, optionally, comprises a detergent.

10. The dual pad of claim 9, wherein the added chase buffer wets the specimen sample collected on the first hydrophilic sample pad and the second hydrophobic sample pad.

11. The dual pad of claim 1, wherein the first hydrophilic sample pad and the second hydrophobic sample pad are in direct contact and, optionally, wherein the first hydrophilic sample pad and the second hydrophobic sample pad overlap in part.

12. The dual pad of claim 1, wherein the first hydrophilic sample pad, the second hydrophobic sample pad, and the third conjugate pad are in direct contact and, optionally, wherein the first hydrophilic sample pad, the second hydrophobic sample pad, and the third conjugate pad overlap in part.

13. The dual pad of claim 1, wherein the first hydrophilic sample pad and the second hydrophobic sample pad are separable or combinable from the third conjugate pad.

14. The dual pad of claim 1 further comprising a unitary cassette and, optionally, wherein at least the first hydrophilic sample pad of the dual pad is located on an exterior surface of the unitary cassette.

15. The dual pad of claim 14, wherein at least the first hydrophilic sample pad of the dual pad provides an exposed surface which protrudes outward from the unitary cassette.

16. The dual pad of claim 14, wherein the unitary cassette comprises a test membrane and, optionally, a viewing window to observe the results shown on the test membrane.

17. The dual pad of claim 1 further comprising a two-part cassette and, optionally, wherein the two-part cassette comprises at least one of a dual pad for fixed volume direct specimen collection comprising a first hydrophilic sample pad and a second hydrophobic sample pad in a primary part or wherein the two-part cassette comprises an opening or internal mechanism to introduce chase buffer to the first hydrophilic sample pad of the dual pad in either the primary part or a secondary part.

18. The dual pad of claim 17, wherein at least the first hydrophilic sample pad of the dual pad is located on an exterior surface of the primary part of the two-part cassette, or wherein at least the first hydrophilic sample pad of the dual pad of the primary part provides an exposed surface which protrudes outward from the two-part cassette.

19. The dual pad of claim 17, wherein chase buffer is added to the first hydrophilic sample pad of the dual pad via an internal mechanism such as a ruptured blister pack.

20. The dual pad of claim 17, further comprising a third conjugate pad in either the primary part or a secondary part of the two-part cassette, or further comprising a test membrane in either the primary part or a secondary part of the two-part cassette.

21. The dual pad of claim 17, further comprising a viewing window to observe the results shown on the test membrane in either the primary part or a secondary part of the two-part cassette.

22. A method for direct collection of a fixed specimen volume comprising the direct introduction of a specimen to the dual pad of claim 1.

23. A kit comprising the dual pad of claim 1, and one of a separable chase buffer container and instructions for use or a cassette comprising a blister pack, and instructions for use.
